(19) United States
(12) Patent Application Publication
(10) Pub. No.: US 2011/0036661 A1
(43) Pub. Date: Feb. 17, 2011

(54) Earpiece Member

(76) Inventor: Ian Edmund Munro, Kent (GB)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/936,890

(22) PCT Filed: Apr. 9, 2009
(86) PCT No.: PCT/GB09/00928

§ 371 Date: Oct. 7, 2010

(30) Foreign Application Priority Data
Apr. 10, 2008 (GB) 0806538.5

(51) Int. Cl. A61F 11/08; B29C 45/00

(52) U.S. Cl. 181/135; 264/299

(57) ABSTRACT

An ear tip or ear plug (20) comprises a body portion (21) having flexible peripheral rib elements (23, 24, 25) projecting therefrom to provide a comfortable and secure fit in a wearer's ear. The rib elements are between 0.2 mm and 2 mm high, preferably 1 mm. The body portion (21) is also flexible and may be made integrally with the rib elements by an injection-moulding process. The body portion (21) has a generally bulbous shape, having a substantially elliptical cross-section and a longitudinal axis (28) which is curved or bent to one side. A conduit (30) may extend between exterior end (38) and interior end (39).

The present invention relates to an earpiece member. In particular it relates to a generic earpiece member, i.e. not one which is custom-made for a particular user. The member may be the ear tip of a communications device, e.g. for radio or telephone communications, or of a device to assist hearing, e.g. a hearing aid. In such devices, it is useful to prevent or at least attenuate the passage of sound around the earpiece, i.e. between the earpiece and the ear of a wearer. This enables the wearer to better hear the sound signals being delivered through a central passage of the earpiece. Alternatively the member may be an ear plug, an aim of which is to substantially prevent or attenuate the passage of sound into a wearer's auditory channel.

Existing earpiece members have relatively rigid bodies having a straight central axis. U.S. Pat. No. 5,002,151, for example, discloses a hearing aid earpiece which is of a generally tubular configuration. Although the earpiece has a sleeve of soft polymer foam to allow it to be compressed to fit in a wearer's ear, the relatively stiff material of the core of the earpiece means that it retains its overall shape. Some existing ear tips have a straight central tubular body portion surrounded by one or more spaced circular discs or flanges and/or one or more projections having an umbrella like configuration.

WO2005/063155 discloses an ear plug of generally frusto-conical shape having ribs protruding from its tapering surfaces. U.S. Pat. No. 4,023,647 discloses an ear plug comprising a generally cylindrical core surrounded by flexible ridges and having a tapered tip.

The relatively rigid material and/or straight body portion of the earpiece members has the problem that they do not conform to the shape of the wearer's ear and thus are uncomfortable to insert and to wear. Another problem is that the external discs or other projections do not efficiently prevent the passage of sound around the earpiece member.

The above problems can be mitigated by producing a custom-made earpiece member for a user, which involves taking a mould of the user's ear. U.S. Pat. No. 6,595,317, for example, discloses a custom-moulded ear-plug device of generally conical shape which may have external reinforcing ribs. However, making a custom mould is a relatively expensive, inconvenient and time-consuming process. In some situations, it is not possible to arrange in advance for a mould to be made.

Aspects of the present invention seek to overcome or reduce one or more of the above problems.

According to a first aspect of the present invention there is provided an earpiece member comprising a body portion at least partly covered with flexible rib elements, wherein the rib elements project from the surface of the body portion by a distance of between 0.2 mm and 2 mm.

The rib elements preferably project by a distance of between 0.5 mm and 1.5 mm and most preferably by substantially 1 mm.

The flexible rib elements are readily deformable by insertion into a wearer's ear.

The body portion is suitably shaped to match the anatomy of a typical human ear, i.e. to fit the auditory meatus.

The ribs may be 0.2 mm to 0.8 mm thick, more preferably 0.4 mm to 0.6 mm thick and most preferably substantially 0.5 mm at their thickest portion, and between 0.2 mm and 2 mm high, more preferably between 0.5 mm and 1.5 mm high and most preferably substantially 1 mm.

The body portion of the earpiece member is also preferably of flexible material. This enables the entire earpiece member to deform to comfortably fit a wearer's ear.

The flexibility of the rib elements and preferably of the entire tip lies within the range 25 Shore A to 85 Shore A.

In preferred embodiments the rib elements are integral with the body portion. This simplifies manufacture. The whole earpiece member is preferably produced in an injection moulding operation.

Preferred materials for the earpiece member are thermoplastic elastomers and/or silicone.

The body portion preferably has a longitudinal axis which is curved or bent. This enables it to fit better in a typical human ear.

There are preferably two to four rib elements. This enables sufficient spacing to be provided between the rib elements while maintaining the rib elements on the central region of the body portion.

The body portion preferably has a substantially elliptical cross-section. The cross-sectional shape may vary slightly along the length of the body portion to enable it to better match a typical wearer's ear. In a preferred embodiment the cross-sectional area of the body portion is smaller towards each end of the body portion than at a central region thereof.

According to a second aspect of the present invention, there is provided an earpiece member comprising a body portion shaped generally to match the anatomy of a typical human ear and at least partly covered with flexible rib elements, wherein the rib elements project from the surface of the body portion by a distance corresponding to 5% to 20% of the width of the body portion.

According to a third aspect of the present invention, there is provided an earpiece member of flexible material comprising a body portion having a longitudinal axis which is curved and two to four rib elements surrounding the body portion transversely of the longitudinal axis.

The rib elements preferably project from the body portion by between 0.2 mm and 2.0 mm.

The rib elements preferably define ellipses.

According to a fourth aspect of the present invention, there is provided an earpiece member comprising a body portion at least partly covered with flexible rib elements, wherein the body portion has a longitudinal axis which is curved and wherein the cross-sectional area of the body portion transversely of the longitudinal axis is smaller towards each end of the body portion than at a central region thereof.

The cross-sectional shape of the body portion is preferably substantially elliptical.

According to a fifth aspect of the present invention, there is provided a method of mass producing earpiece member without making moulds from wearers' ears, the method employing a mould which defines a shape having a body portion with a longitudinal axis which is curved and two to four rib elements surrounding the body portion transversely of the longitudinal axis.

The method is preferably an injection-moulding method. This has the advantage of being quick and convenient. The mould is preferably configured to produce a hollow earpiece member.

Preferred embodiments of the present invention will now be described, by way of example only, with reference to the accompanying drawings, of which:

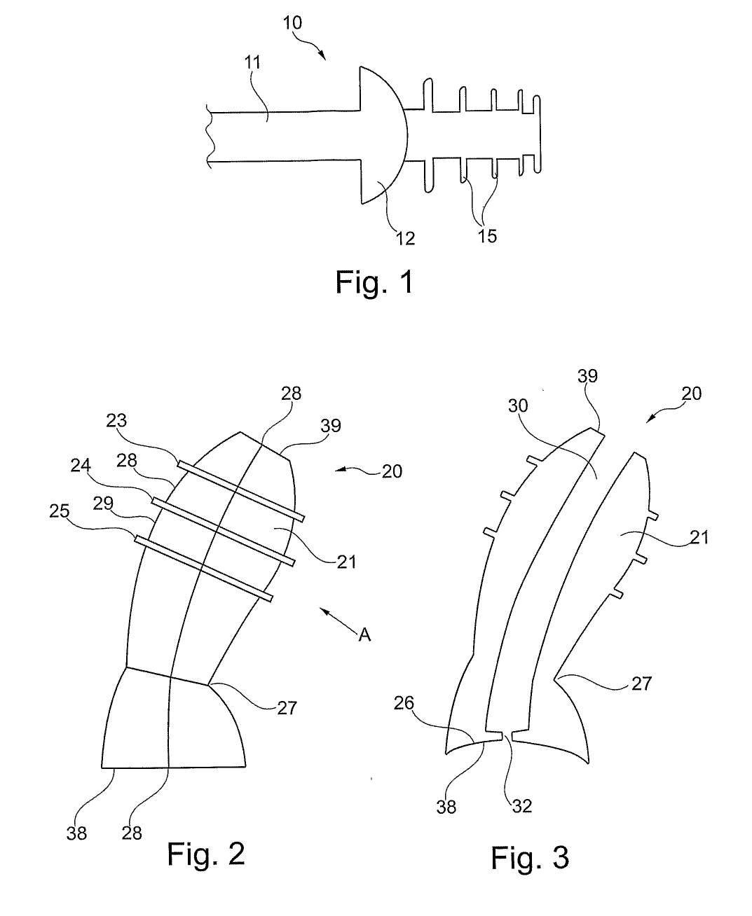

FIG. 1 shows an ear tip according to the prior art;

FIG. 2 shows a side view of an ear tip according to a first embodiment of the present invention;

FIG. 3 shows a cross-sectional side view of the ear tip of FIG. 2;

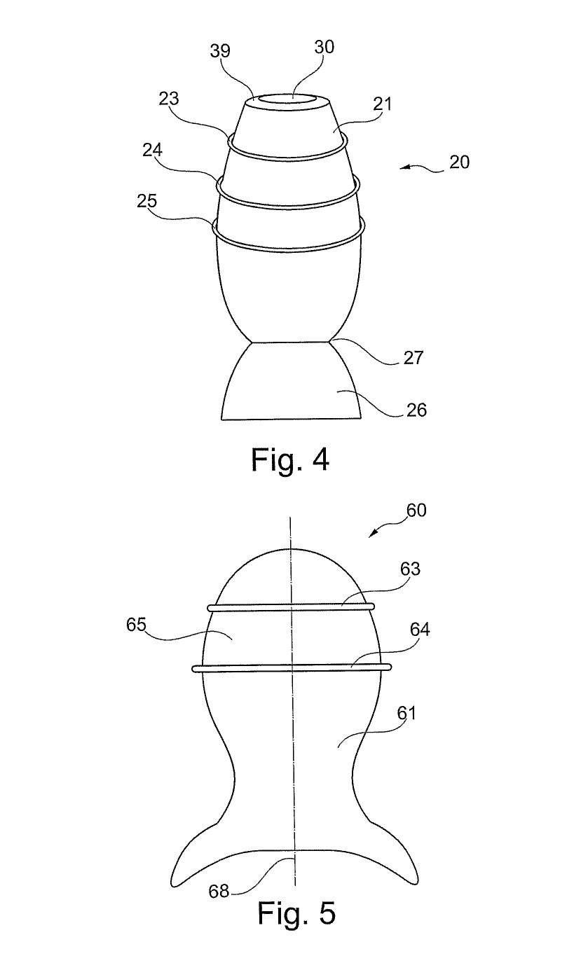

FIG. 4 is a front view of the ear tip of FIGS. 2 and 3 in the direction of the arrow A in FIG. 2; and

FIG. 5 is a view corresponding to FIG. 2 of an ear tip in accordance with a second embodiment of the present invention.

An ear plug or ear tip mould is intended to plug the ear canal of a wearer such that the ingress of sound or air or liquid is prevented between the exterior surface and the interior surface of the ear. The device may be solid or hollow or a combination of both.

The device may have a conduit connecting the exterior end to the interior end such that sound or light or communications or electricity or liquid or gas or any combination may pass through. Alternatively the device may be solid or fitted with an acoustic or other attenuator to restrict the conduit.

The conduit may be fitted with a plug or tube or filter or metal conductor, for the purpose of conveying or restricting sound or light or liquid or gas or electricity.

Referring to the drawings, FIG. 1 shows an ear tip 10 in accordance with the prior art. The tip comprises a cylindrical core 11 surround by an umbrella-like projection 12 and a plurality of disc-like projections 15 of gradually reducing diameter along the core. The diameter of the core is approximately 5 mm and each disc projects by approximately 3 mm from the surface of the core. Thus the core and the projections share the function of occupying the ear canal, i.e. a substantial part of the cross-section of the ear canal is occupied by the discs 15 themselves. Although the umbrella portion 12 and to a lesser extent the discs 15 may deform slightly on insertion into an ear, the overall straight shape of the ear tip does not change to adapt itself to the wearer's ear. Despite the slight deformation of the projections 12, 15, the ear tip is usually uncomfortable to wear for a long period of time.

FIGS. 2 to 4 show an injection-moulded ear tip 20 in accordance with a first embodiment of the present invention. The tip is of a general ergonomic shape to fit the anatomy of an ear or ear canal so as to be capable of plugging it. The tip is made from an elastomeric or flexible or deformable material such as plastics or rubber. In particular, the ear tip can be made of a thermoplastic elastomer or a silicone, e.g. a thermo-setting silicone. Thus the tip is designed to substantially match the shape of a wearer's ear so that it is comfortable to wear, even over long periods of time.

The body portion 21 of the ear tip is of a size such that it occupies the predominant part of the cross-section of the ear canal. It has a generally bulbous shape to match the internal shape of a generic human ear. Over the surface of the body portion, at least towards its end arranged to be inserted in an ear, there are provided three rib or fin elements 23, 24, 25. The rib elements have a thickness or an average thickness of 0.5 mm and a height or an average height of 1.0 mm. The longitudinal axis 28 of the body portion is curved and the rib elements are arranged substantially perpendicular to the longitudinal axis 28. The rib elements 23, 24, 25 extend all the way around the external surface of the body portion 21. After insertion of the ear tip in the ear, the rib elements define between them enclosed pockets 28, 29 which are approximately annular. It will be noted that the height of the rib elements is substantially smaller than the diameter of the body portion, so that they occupy only a small proportion of the cross-section of the ear canal. They serve to make the tip comfortable to wear and convert a body portion, which might only provide a poor fit, into an ear tip, which provides a snug and effective fit. The rib elements 23, 24, are conveniently formed at the same time as the body portion and of the same material in a single injection-moulding process.

At the end of the body portion 21 adjacent to the exterior end of the earpiece member, there is provided a slightly-dished widening portion 26 (see FIG. 4) meeting the body portion at a neck 27. The dished portion 26 has a width of 10.0 mm, a height of 1.5 mm and a radius of 5.0 mm, see FIG. 3. The width of the tip at neck 27 is 6.5 mm.

The tip 20 has a conduit 30 connecting exterior end 38 to interior end 39, see FIG. 2. Where it emerges at end 39, conduit 30 has a diameter of 2.5 mm. Conduit 30 has a narrow mouth 32 at the exterior end. A communications device can be connected to mouth 32. Alternatively a sound attenuating plug can be inserted into the mouth 32 to vary the effect of the ear tip. The shape and size of the conduit 30 is arranged to substantially follow the outer contour of the tip 20. This provides a hollow section and allows the initial or natural shape of the tip to deform, especially to fit unusually-shaped ear canals.

The thickness of the body portion 21 between its outer surface and conduit 30 is substantially greater than the height of the ribs.

In use, as the ear tip 20 is inserted in a wearer's ear, the rib elements 23, 24 are deformed against the surface of the ear or ear canal and form an effective seal therewith against the ingress of sound. This has the advantage of enhancing the effectiveness of the ear tip in attenuating sound.

The effectiveness of the seal is believed to be due to the formation of a plurality of separate pockets of air corresponding to the spaces between the individual rib elements. There are no direct paths for sound signals, which are instead heavily attenuated as they transverse the barriers constituted by the rib elements 23, 24, 25. The seal is also liquid tight and substantially gas tight.

The rib elements also serve as an all round cushion between the earpiece and the ear or ear canal. This enhances the comfort of a wearer; the flexible body portion 21 substantially deforms to match the shape of a wearer's ear and the rib elements deform to make the resulting fit comfortable for the wearer.

The approximate cross-sectional shape of the tips is preferably elliptical or circular but a wide range of shapes are possible to suit individual wearer's ears, and the shape may vary along the longitudinal dimension of the tip.

For the four types of tip, indentified as 1#, 2#, 3# and 4#, the preferred dimensions A, B and C are given in Table 1 below, wherein:

A is the longitudinal dimension of the tip

B is the maximum transverse dimension of the tip, e.g. the major diameter of the ellipse; and

C is the minimum transverse dimension of the tip at its widest part, e.g. the minor diameter of the ellipse where its cross-sectional area is greatest along the longitudinal dimension.

TABLE 1

| Tip Type | Dimension | Size in mm |
|---|---|---|
| 1# | A | 14.5 |
| 1# | B | 6.0 |
| 1# | C | 7.0 |
| 2# | A | 15.5 |
| 2# | B | 7.0 |
| 2# | C | 8.0 |
| 3# | A | 16.5 |
| 3# | B | 8.0 |
| 3# | C | 8.0 |
| 4# | A | 17.5 |
| 4# | B | 8.0 |
| 4# | C | 9.0 |

The rib elements 23, 24, 25 are preferably spaced about 2 mm apart. Each rib element has a rounded peak so that it has no sharp edges. Each rib element may have a thickness or an average thickness of 0.3 mm to 0.8 mm and a height or an average height of 0.2 mm to 2.0 mm, preferably 0.5 mm to 1.5 mm.

The ear piece member 20 is conveniently made by an injection-moulding process. In such a process it is simple to produce a conduit 30 of any desired shape. For custom-made earpieces, production of a conduit can be a complicated process. The present invention allows a large plurality of identical earpiece members to be quickly produced.

Numerous modifications may be made to the above-described earpiece member.

The rib elements may be produced in any convenient manner, e.g. by forming the body portion and then cutting or otherwise producing recessed areas 28, 29 therein; however this is a relatively lengthy procedure. Alternatively the rib elements 23, 24, 25 may be moulded on or adhered to the surface of the body portion 21 in a separate manufacturing step. This allows the body portion and the rib elements to be of different materials; for example, the body portion can be of a relatively rigid material, but preferably a foamed or cellular version thereof so as to maintain some flexibility for adapting to the shape of a wearer's ear.

The rib elements may form any desired pattern or, indeed, no pattern at all. They may be arranged diagonally or helically. They may be arranged to form intersecting sets to form a plurality of relatively small pockets. One set may be substantially as shown with the other set extending longitudinally of the body portion. Alternatively, both sets may extend diagonally or contra-helically, i.e. forming intersecting helices, or in a honeycomb or other regular pattern. The intersecting sets of rib elements may be mutually perpendicular or not. In general the rib elements may comprise annular and/or peripherally extending elements to form any suitable grid or lattice of cellular construction or continuous peripheral rings. If desired the rib elements may extend over the entire surface of the body portion, but the outside end 38 of the ear tip is preferably left free of rib elements for ease of handling.

In an alternative modification, the body portion 21 is covered with a plurality of sinuous rib elements to form enclosed areas with various curved shapes.

Although preferably forming a continuous pattern, the rib elements themselves do not need to be continuous. They may have any suitable cross-sectional shape; for example they may be of uniform width and have square tops.

Conduit 30 may be omitted so that an ear plug is provided.

The body portion 21 may be generally cylindrical, ellipsoidal or conical in shape and its longitudinal axis may be curved or bent as desired to match the internal shape of an ear.

FIG. 5 shows an ear tip 60 in accordance with a second embodiment of the present invention. Here, body portion 61 has only two rib elements 63, 64 provided thereon, arranged at right-angles to the longitudinal axis or dimension 68 of the ear tip. Although, after insertion in an ear, only a single pocket is formed above the area 65 between the rib elements, reasonable sound attenuation is still provided in addition to a comfortable fit. The spacing between the rib elements is between 1 mm and 8 mm. As shown, ear tip 60 is rotationally-symmetrical about its longitudinal axis 68, i.e. it is straight. Alternatively, its longitudinal axis can be curved or bent at an angle to match it better anatomically to the human ear.

The features and modifications of the various embodiments and aspects of the invention may be combined or interchanged as desired.

1.-13. (canceled)

14. An earpiece member (20, 60) comprising a body portion (21, 61) at least partly covered with flexible rib elements (23, 24, 25; 63, 64), wherein the rib elements project from the surface of the body portion by a distance of between 0.2 mm and 2 mm, characterised in that the longitudinal axis of the body portion (21) is curved.

15. An earpiece member according to claim 14, wherein the body portion (21, 61) has a substantially elliptical cross-sectional shape.

16. An earpiece member according to claim 14, wherein the body portion (21, 61) is made of flexible material.

17. An earpiece member according to claim 14, wherein the rib elements (23, 24, 25; 63, 64) surround the body portion (21, 61) transversely of the longitudinal axis.

18. An earpiece member according to claim 17, wherein there are two to four rib elements (23, 24, 25; 63, 64).

19. An earpiece member according to claim 18 comprising two rib elements (23, 24; 63, 64) mutually spaced by between 1.5 mm and 5 mm.

20. An earpiece member according to claim 18 comprising three rib elements (23, 24, 25) mutually spaced by between 1.5 mm and 5 mm.

21. An earpiece member according to claim 14, wherein the cross-sectional area of the body portion (21, 61) is smaller towards each end of the body portion than at a central region thereof.

22. An earpiece member (20, 60) comprising a body portion (21, 61) with a longitudinal axis which is curved, the body portion being at least partly covered with flexible rib elements (23, 24, 25; 63, 64), wherein the rib elements project from the surface of the body portion by a distance corresponding to 5% to 20% of the width of the body portion.

23. An earpiece member according to claim 22, wherein the body portion (21, 61) has a substantially elliptical cross-sectional shape.

24. An earpiece member according to claim 22, wherein the body portion (21, 61) is made of flexible material.

25. An earpiece member according to claim 22, wherein the rib elements (23, 24, 25; 63, 64) surround the body portion (21, 61) transversely of the longitudinal axis.

26. An earpiece member according to claim 25, wherein there are two to four rib elements (23, 24, 25; 63, 64).

27. An earpiece member according to claim 26 comprising two rib elements (23, 24; 63, 64) mutually spaced by between 1.5 mm and 5 mm.

28. An earpiece member according to claim 26 comprising three rib elements (23, 24, 25) mutually spaced by between 1.5 mm and 5 mm.

29. An earpiece member according to claim 22, wherein the cross-sectional area of the body portion (21, 61) is smaller towards each end of the body portion than at a central region thereof.

30. A method of mass producing ear members (20, 60) without making moulds from wearers' ears, the method employing a mould which defines a shape having a body portion with a longitudinal axis which is curved and two to four rib elements surround the body portion transversely of the longitudinal axis.

31. A method according to claim 30, wherein the shape of the body portion defined by the mould has a cross-sectional area which is smaller towards each end of the body portion than at a central region thereof.
